(19) United States
(12) Patent Application Publication
(10) Pub. No.: US 2015/0313534 A1
(43) Pub. Date: Nov. 5, 2015

(54) Rules-Based Message Prioritization or De-Selection with Patient Glucose Monitors

(71) Applicant: EosHealth, Inc., Houston, TX (US)

(72) Inventor: Kimon J. Angelides, Houston, TX (US)

(21) Appl. No.: 14/338,221

(22) Filed: Jul. 22, 2014

Related U.S. Application Data
(63) Continuation of application No. 14/307,906, filed on Jun. 18, 2014.
(60) Provisional application No. 61/988,179, filed on May 3, 2014.
(60) Provisional application No. 61/993,373, filed on May 15, 2014.

(51) Int. Cl. A61B 5/00; A61B 5/0205; A61B 5/145

(52) U.S. Cl. CPC A61B 5/486; A61B 5/14532; A61B 5/0205; A61B 5/14546; A61B 5/4866; A61B 5/4848; A61B 5/4833; A61B 5/7282; A61B 5/74; A61B 5/021

(57) ABSTRACT

An interactive diabetes monitoring and advice system includes a patient glucometer device with a wireless link to a server which can provide messages to the patient, such as advice and educational messages. The messages are associated such that selection of certain advisory messages or blood glucose levels, or exertion levels, leads to sending of certain educational or other messages, while certain other educational or other messages are not sent, or not sent for a specified period.

### BACKGROUND

As one of America's deadliest diseases, and as there are over 20 million American diabetics, diabetes mellitus places a particularly high expense burden on the public healthcare system. Millions of Americans are not even aware that they have the disease, and an additional 50 million plus Americans have pre-diabetes. If the present trends continues, 1 in 3 Americans, including as many as 1 in 2 minorities born in 2000 will develop diabetes during their lifetime.

Diabetes is a group of chronic metabolic diseases marked by high levels of blood glucose resulting from defects in insulin production, insulin action, or both. While diabetes can lead to serious complications and premature death, effective treatment requires the diabetic patient to take steps to control the disease and lower the risk of complications.

Uncontrolled diabetes leads to chronic end-stage organ disease and in the United States is a leading cause of end-stage renal disease, blindness, non-traumatic amputation, and cardiovascular disease. It is also associated with complications such as:
Heart Disease and Stroke (#1 cause of death for diabetics and 2-4 time higher than the general population)
High Blood Pressure (3 in 4 diabetics)
Nervous System Damage (can lead to amputations and carpel tunnel syndrome)
Pregnancy Complications (including gestational diabetes)
Sexual Dysfunction (double the incidence of erectile dysfunction)
Periodontal Disease

In the USA, over 85% of people aged 65 and over have diabetes, a fact that complicates their total health picture and often accelerates chronic end-stage disease, adding an enormous strain to the healthcare system. In addition, there are correlations of higher diabetes incidence with smokers, and Alzheimer's patients.

Poor control of blood-glucose in diabetes dramatically increases the risk of heart disease, stroke, amputations, blindness, renal disease and failure, impotence, and many other diseases—better control of blood-glucose levels greatly mitigates these complications. Coupled with proper education, nutrition, maintenance of stable blood-glucose levels, and regular exercise, many Type 1 and 2 diabetics can minimize the effects of the disease.

With the growing problem of diabetes in developed and developing countries comes a growing need for convenient blood glucose monitoring, and convenient methods for analysis and treatment based on the monitoring. Diabetics need to monitor their blood glucose multiple times a day and record this information, which is analyzed, along with other parameters such as quantity of exercise and their diet, and then use the results to determine food intake, adjust the dosage of insulin and/or other therapeutic agent, and to recommended exercise intensity or cessation. Compliance with the monitoring, diet and exercise regimes is a challenge due to their complexity and temptation to avoid the recommended diet, which is low in simple sugars, and the recommended exercise regime.

A hand-held, portable wireless device, linked to and interactive with a server and with personal health monitors for the user, can be used assist in compliance by reminding the patient of the need to test periodically, by logging the blood glucose test results and the associated meal information and the carbohydrates ingested and the patient feelings, (and storing the results in a user friendly display form as averages and other analysis), and also by providing selected advisory and educational messages, and providing sharing with select health monitors and other selected parties, all with the aim to increase compliance with the recommended monitoring, diet and exercise regimes.

### SUMMARY

Preferred user devices and interactive systems for use with the invention include those described in U.S. Pat. No. 8,066,640 and US Publ'n No. 20130035563 (both of which are incorporated by reference). In brief, these references together describe a system of education, monitoring and advising on glucose testing, diet, exercise and drug administration using a device which is lightweight and portable (and easily carried by the patient) and which is capable of: blood glucose testing, displaying messages advising the patient to initiate blood glucose testing, and of recording the results of the test; of displaying advice or further queries based on analysis of the results, including advising for testing ketones if the blood glucose level is above a threshold level; analyzing other blood glucose-related and health-related information and personal information, including patient-identifying information and patient preferences (particularly for diet and exercise) and patient limitations (can't run, for example) which can input by the patient periodically or input and stored; and of displaying advice, education and/or or further queries based on the analysis. US Publ'n No. 20130035563 lists numerous messages in the category of “exemplary educational messages” when in fact many of those messages are advisory messages as defined herein, while others are in another of the four categories in Table II below.

As the device's computing power or access to full patient information is limited, and because the ability of health care professionals to provide advice is also desired, the device is preferably linked wirelessly to a server that performs some or all of the analysis and information storage described above. In the case of employing a server, the glucose test results and preferably also information about food intake, exertion and patient feelings and symptoms, are transmitted to the server. The device receives the results of the server's analysis in the form of queries, advice and educational messages. The wireless link to the device also provides the ability for feedback, advice and/or intervention from appropriately experienced health care workers, as necessary and appropriate.

The device preferably also includes the ability to test ketone levels and record the results, track timing of food consumption and foods, particularly carbohydrates, consumed, and a pedometer or accelerometer to track patient exertion and estimate total calories expended in exercise.

The analysis from the server is then used to select from a bank of messages (containing at least 100 educational messages and at least 100 advisory messages) to send to the device (and the user). The messages relate to advice on further testing, food consumption and exertion, as well as general diabetes education, and are preferably suitable for display on a small screen, typical of a hand-held device—meaning the messages are necessarily compact.

There are two broad categories of messages sent to patients from a message bank (which holds hundreds of messages of each type): advisory and educational. Advisory messages can include:

(i) advising the patient to test for chemical or biochemical indicators, including blood glucose level, HbA1c level, LDL level, ketone level, in vivo drug or insulin concentration, blood pressure, or gene expression level;
(ii) advising the patient to take action with respect to eating or exercising, e.g., recommending specific food items or types for intake, recommending a time for food intake, recommending ceasing or commencing exercise; and
(iii) advising the patient to take the less-frequently required actions for health maintenance, including general medical check-ups (by a physician), eye check-ups, checking of feet and lower legs for wounds, checking of any non-healing wounds.

Educational messages do not recommend immediately taking specific actions but advise the patient generally of risks and benefits of actions, indications or inaction (e.g., failing to maintain desired BG levels).

In the system described, the messages in the message bank selected to be sent to a particular patient can be prioritized if, for example, recommending immediate action if the need for action is acute (e.g., BG level of other indicators are far out of range). Messages can also be prioritized recommending action where the patient's profile is consistent with prioritization, or where the patient is non-compliant with respect to: testing/reporting BG or indicator levels; reporting diet; or monitoring and reporting of other health-related information such as recommended wound, foot and lower leg care, eye care, or scheduled medical check-ups. Messages can also be prioritized based on patient morbidities or risk assessment for morbidities, or whenever such risk assessment changes. Other messages from the message bank can be de-selected, so they are not sent to particular patients or not sent in particular circumstances. Messages can be de-selected based on the patient's profile, to take into account patient's physical limitations such that instructions to “run” would not be sent to disabled patients; instructions praising patient's actions would be de-selected for non-compliant patients. Messages can also be de-selected based on patient morbidities or risk assessment for morbidities, or whenever such risk assessment changes.

### BRIEF DESCRIPTION OF THE DRAWINGS

FIG. 1 depicts a system and algorithm for optimizing messages which are most effective in maintaining the patient's BG level within, or moving it into, a desired range; including where the message is only sent once.

FIG. 2 depicts a system and algorithm for optimizing messages which are most effective in maintaining the patient's BG level within, or moving it into, a desired range.

FIG. 3 depicts a system and algorithm for optimizing messages which are most effective in maintaining the patient's BG level within, or moving it into, a desired range.

FIG. 4 depicts a system and algorithm for optimizing messages which are most effective in maintaining the patient's exertion level within, or moving it into, a desired range.

FIG. 5 depicts a system and algorithm for optimizing messages which are most effective in prompting a user to test their BG level.

### DETAILED DESCRIPTION

The prioritization and de-selection of messages in the message bank can be set based on the information in the patient's personal profile, which is preferably provided by the patient initially. See US Publ'n No. 20130035563 (incorporated by reference). As described in US Publ'n No. 20130035563, the patient's preferences govern the messages sent regarding food, exercise and education. The messages prioritized and de-selected would also be governed by the medications the patient is currently prescribed, as well as the patient's general state of health and risk of co-morbidities (which can include analysis of the patient's genetic profile).

As the personal profile changes over time (e.g., food likes and dislikes may change; exercise preferences and exclusions and physical limitations likely would change; state of general health and co-morbidity risk likely would change; medications also likely would change) the messages which are prioritized or de-selected in the message bank would change in a corresponding manner. For example, messages would not be sent recommending extreme exertion after a heart attack. Messages would not be sent recommending a medication which is no longer prescribed, but messages would be prioritized to recommend taking a newly prescribed medication, as scheduled. Similarly, changes in the state of general health and the co-morbidity risk could result in certain foods, activities and medications being contra-indicated, or more strongly contra-indicated (stopping smoking after a heart attack), and messages could be prioritized to recommend avoidance of such foods, activities and medications.

In the system of prioritization and de-selection of messages described above, prioritization of messages in the message bank can include any of the following: the message is sent once; the message is either sent at a specified frequency for a set period and/or until the requirement it requests is filled; the message is sent at a specified frequency indefinitely. De-selection of messages in the message bank can include any of the following: the message is never sent again; the message is not sent for a specified period and/or until a countervailing concern has been rectified; the message is sent again at specified time(s) and/or frequencies.

Whether or not prioritization or de-selection of certain messages is indicated for a patient, it is also preferable that some messages are more frequently selected from the group of possible messages based on a variety of factors, which can include the patient's individual food and exercise likes and dislikes, physical limitations, schedule limitations, past actions or inactions, metabolic indicators, and likely message effectiveness (based on patient's response to prior messages). The frequency of selection can be controlled a number of ways, including individual weighting of all the messages in the message bank, or weighting of all messages in a particular category, or by a combination of prioritizing and de-selection of certain messages and weighting of others (in particular circumstances).

Circumstances where a combination of prioritizing/de-selecting certain messages and changing the selection frequency of others are appropriate include:

where preferences of the patient change, then certain messages directly relating to reinforcing the new preferences are prioritized and other messages counter to the new preferences are de-selected, and then other messages in the bank can be optimized based on their effectiveness in prompting patient responses, in view of the foregoing changes in the message bank (from prioritization and de-selection);
when BG levels or levels of other chemical or biochemical indicators are out of range, specific advisory messages from categories (i) and/or (ii) (in the Summary) would be prioritized, and certain educational messages (preferably) would be prioritized—i.e., those advising of risks of out of range levels. Other educational messages could also be selected based on other factors, and frequency of sending them can by controlled by an optimization procedure;
when the patient fails to test BG levels or levels of other chemical or biochemical indicators at the recommended interval, advisory messages from category (i) (in the Summary) would be prioritized and preferably sent at intervals until the testing is performed and reported. In addition, educational messages relating to the risks of failing to test as recommended would be prioritized and frequency of sending other educational messages can be controlled by an optimization procedure;
when the patient fails to take the recommended action with respect to eating or exercising (or fails to report that they complied with the recommended diet or exercise actions), advisory messages from category (ii) (in the Summary) would be prioritized and preferably sent at intervals until the action is performed and reported. In addition, educational messages relating to the risks of failing to diet and exercise as recommended would be prioritized. Other educational messages could also be prioritized based on other factors, and frequency of sending them can by controlled by an optimization procedure; and
when the patient fails to take or report the recommended action as set forth in category (iii) (in the Summary), advisory messages from category (iii) would be prioritized and preferably sent at intervals until the action is performed and reported. In addition, educational messages relating to the risks of failing to act as recommended would be prioritized and other educational messages could also be prioritized based on other factors, and frequency of sending them can by controlled by an optimization procedure.

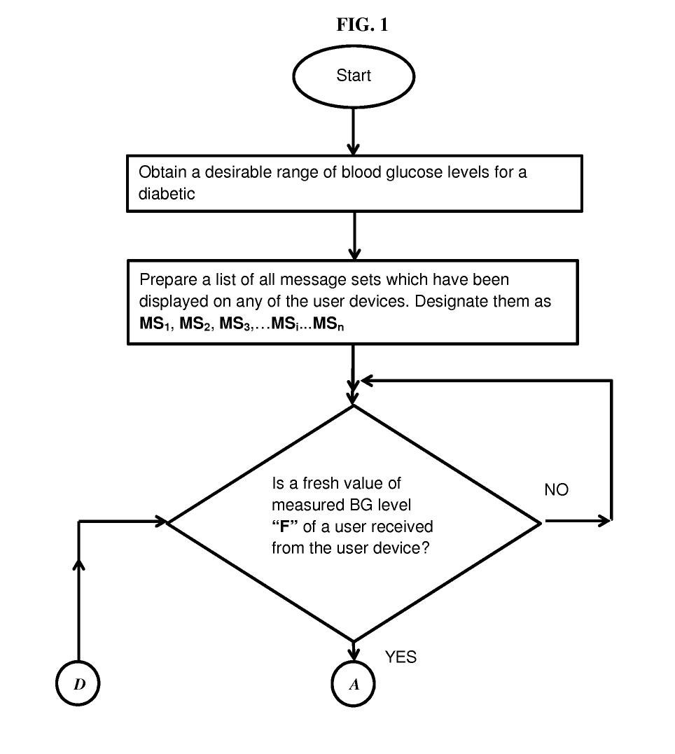

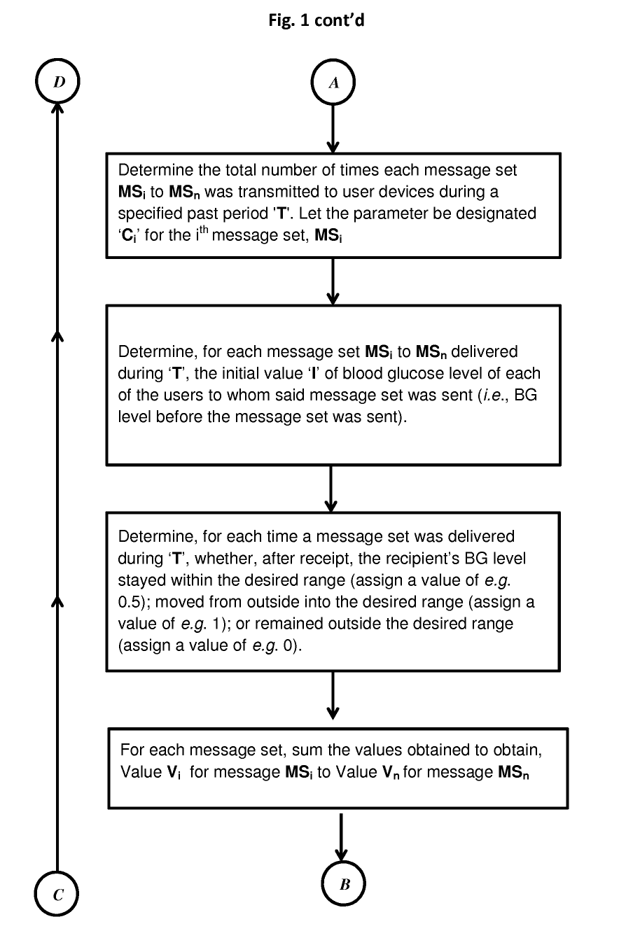

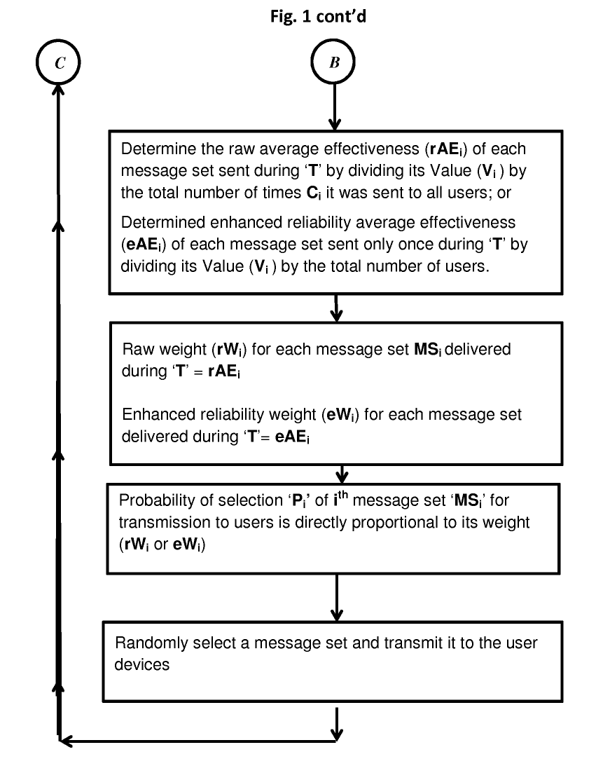

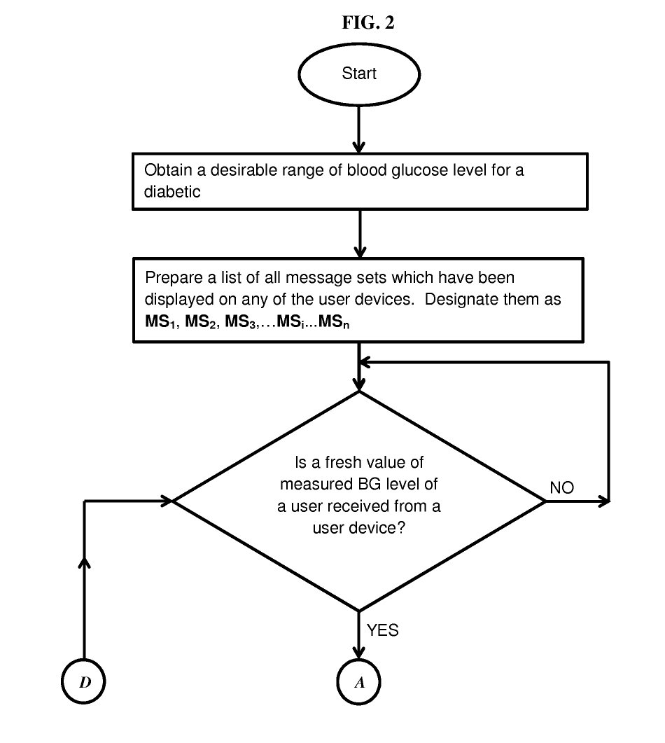

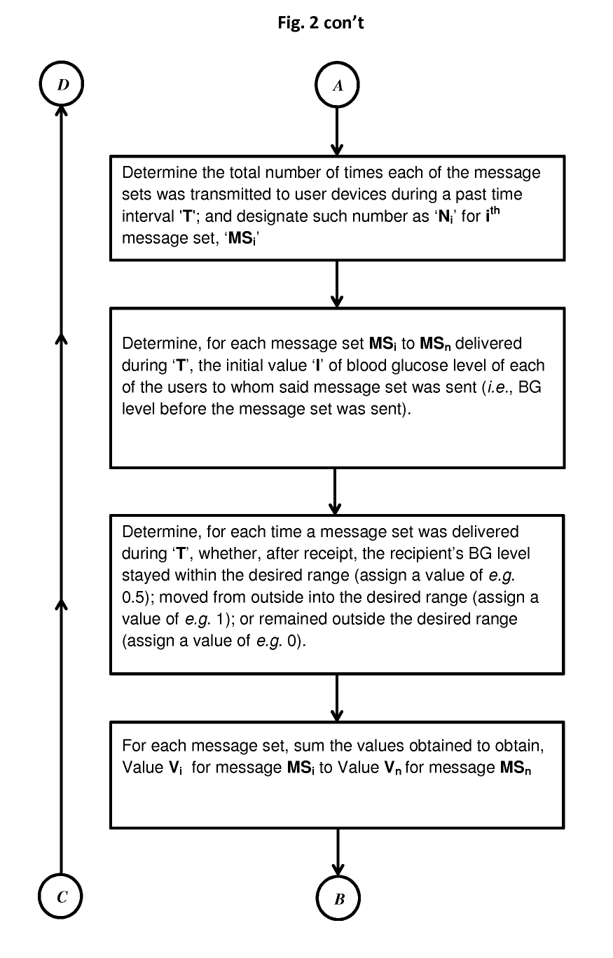

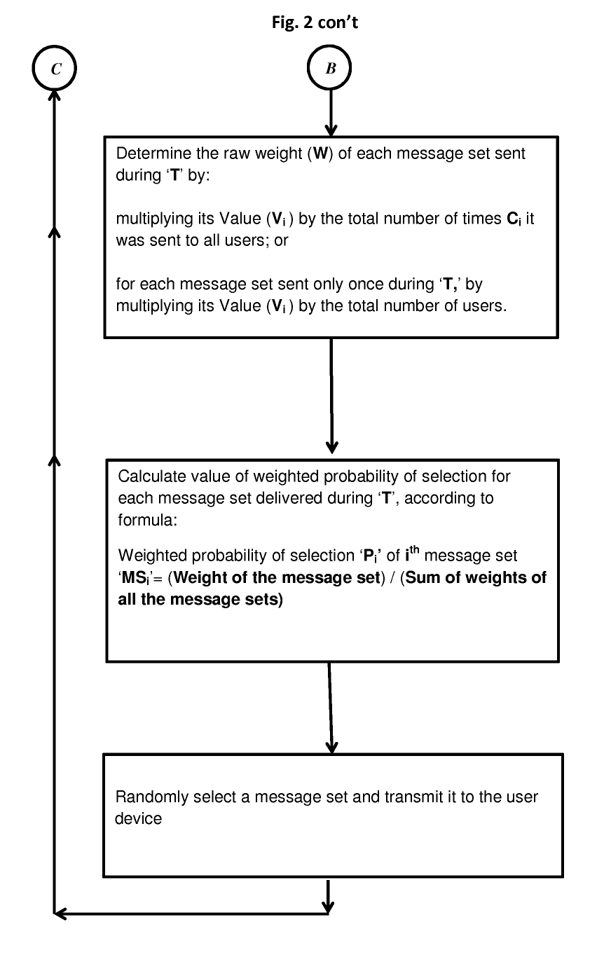

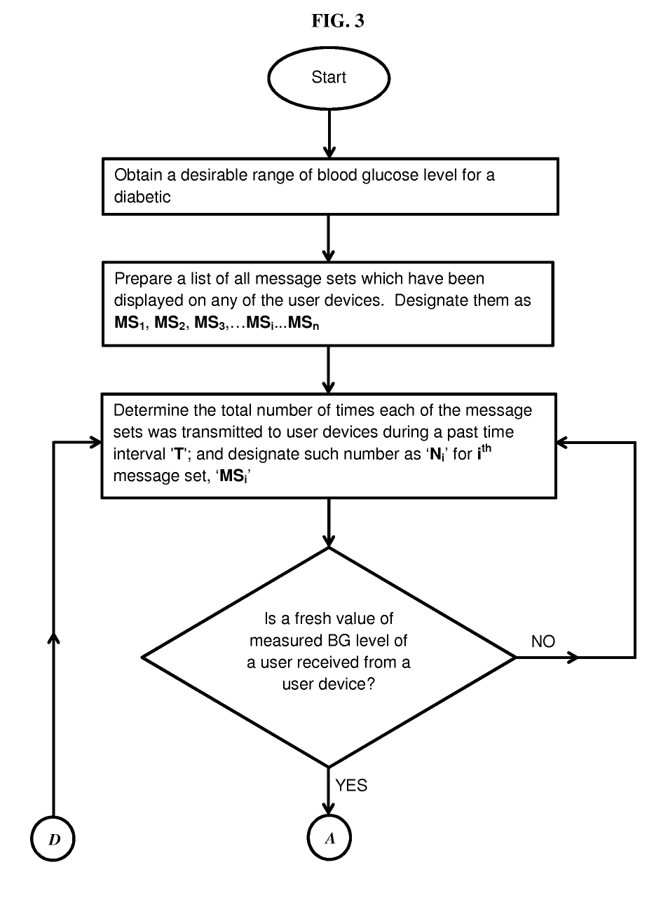

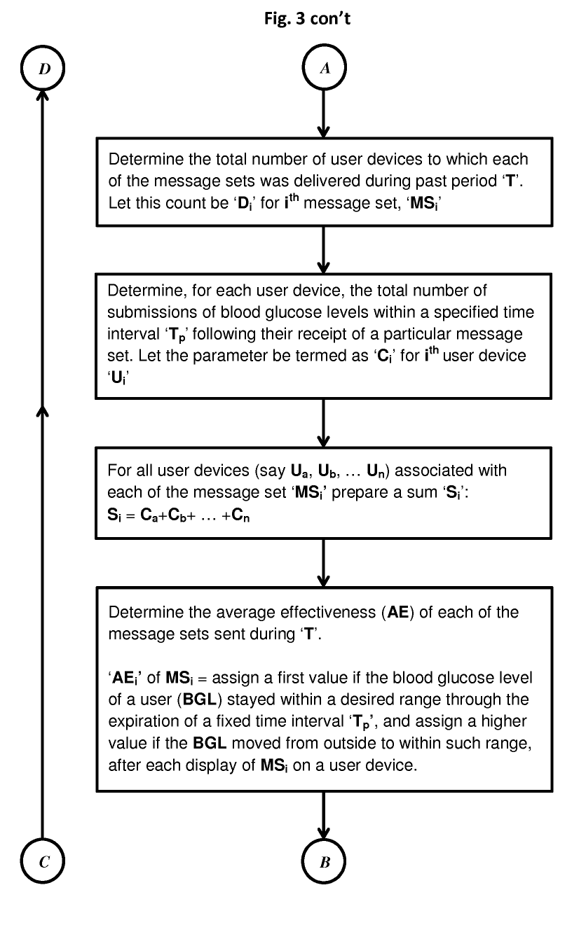

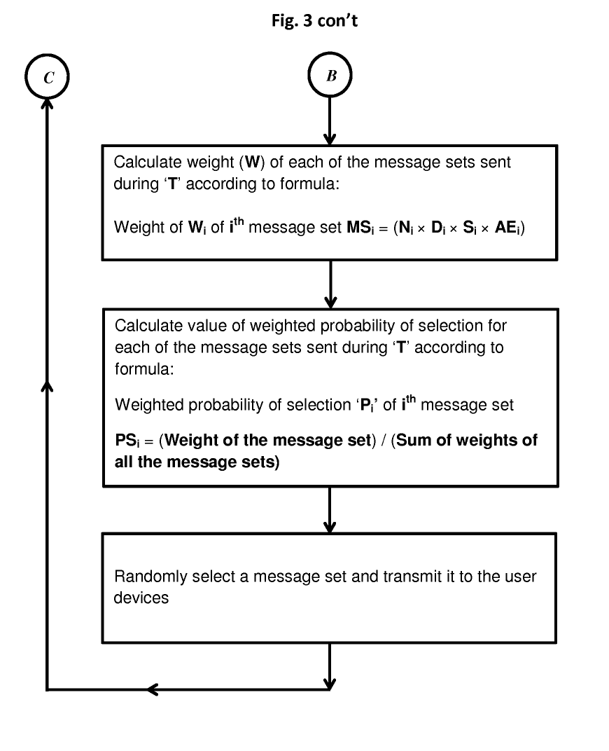

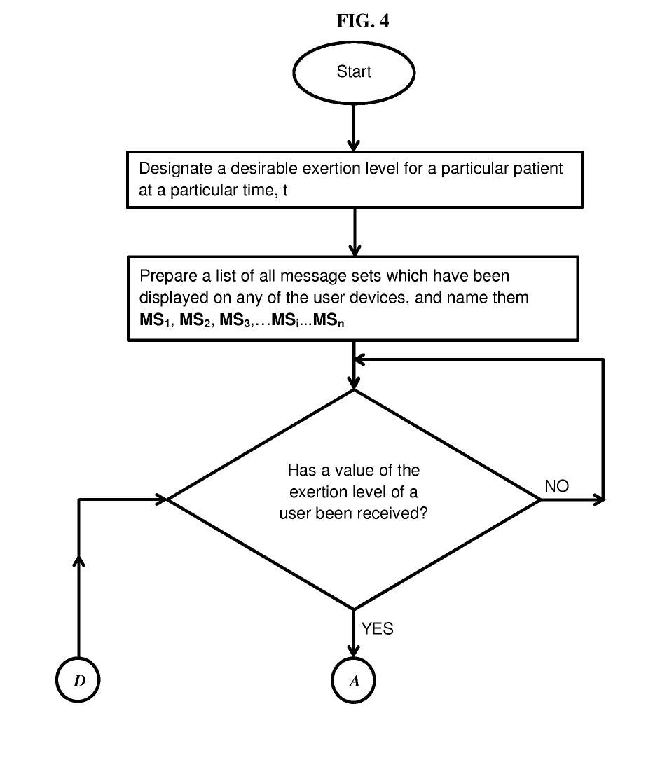

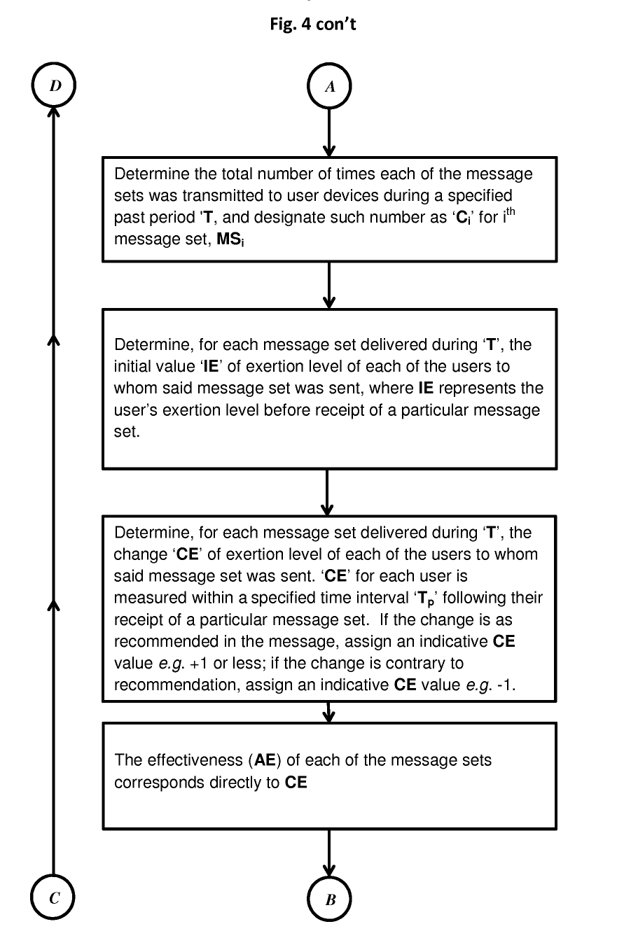

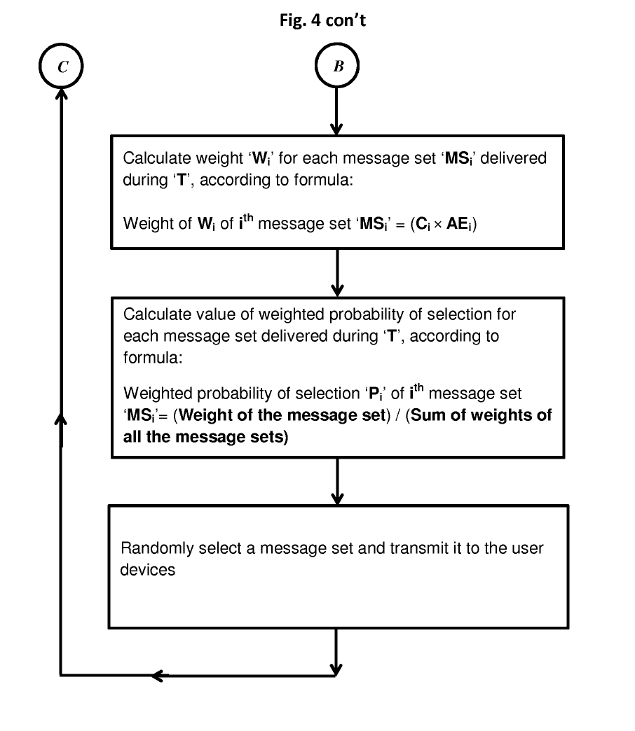

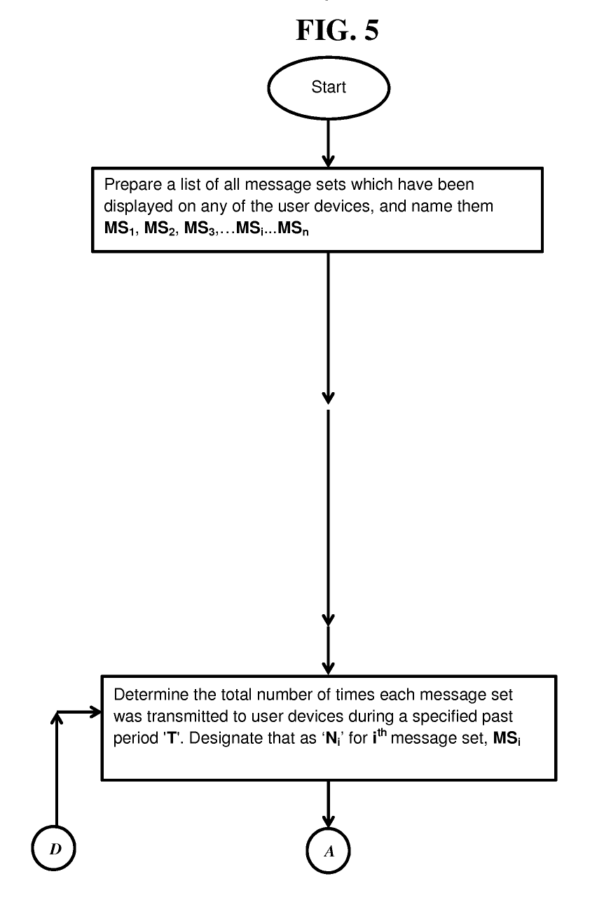

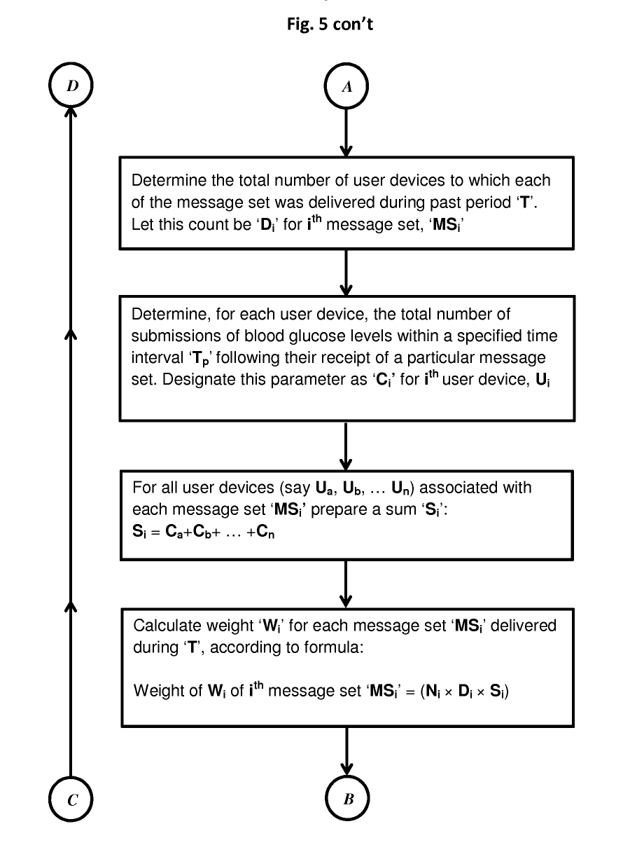

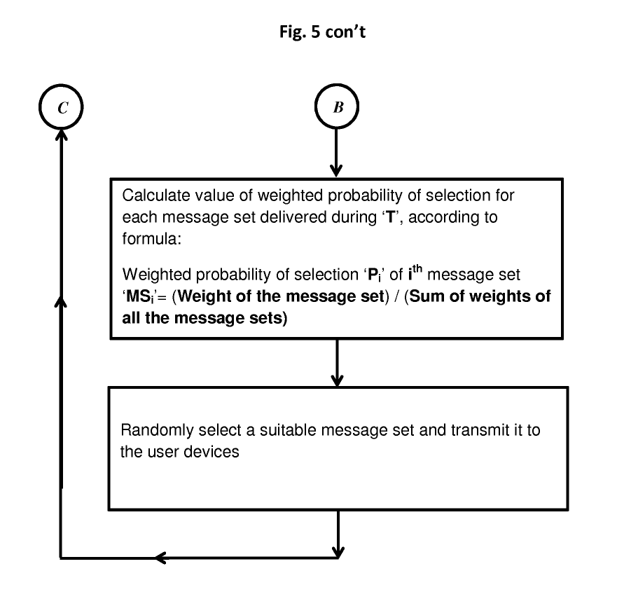

Turning to controlling frequency of message selection using, e.g., optimization through weighting, the weighting of messages (and/or other method of controlling their frequency of selection) can be set initially but is expected to change over time based on the effectiveness of the message in prompting the desired patient response to it (see FIGS. 1 to 5 herein and U.S. application Ser. No. 14/307,906, incorporated by reference). The patient response to messages can be objectively determined based on the response as determined by subsequent BG levels or levels of other indicators, based on patient exertion level (as measured and reported by the patient or as measured and automatically reported by a pedometer or accelerometer carried by the patient), patient diet (as reported by the patient), or based on clinical endpoints including death or diabetic disease markers, including non-healing wounds, hypertension, neuropathy, nephropathy, stroke, gastroparesis, ulcers, heart disease, and cataracts. Such responses can be used to optimize the messages sent to the patient, as described in U.S. application Ser. No. 14/307,906 (where the optimization is achieved through sending a message to users, weighting based on the effectiveness in prompting patient responses desired, randomly selecting the weighted messages and again determining effectiveness, and repeating the cycle so optimization is continuous). See also FIGS. 1 to 5 herein, showing weighting and optimization procedures for optimizing messages relating to control of blood glucose level, of exertion level and of frequency of testing for blood glucose level.

As noted above, prioritization includes increasing the frequency of sending messages, which can be based on any of the factors noted above. In some cases (particularly, where a recommended action is not required for patient health, e.g., changes in food or exercise preferences rather than food or exercise prohibitions) the frequency of sending certain messages can be decreased (a type of de-selection) based on the same factors which lead to message de-selection.

An exemplary table below shows the prioritization and de-selection of messages described above:

TABLE I
Prioritizing and De-Selecting Messages in a Message Bank

For each message: Raise the probability of it being sent; or, lower the probability of it being sent,
by placing it in one or more of the following categories:
Absolute Prioritized messages = Always sent, until_stop sending;
Absolute Deselected messages = Never sent, until_start sending.
Prioritize message frequency: whereby it's sent at frequency X, until _, then change
frequency;
Deselect message frequency: whereby it's sent at frequency no greater than X, until_, then
change frequency.
Raise or lower the frequency of sending a particular message by, e.g., changing the probability of
a particular message being sent by weighting and re-weighting based on effectiveness, or
otherwise optimizing the effectiveness of the messages sent based on one or more of: patient
responses, objective measures of e.g. exertion level, chemical indicators or clinical outcomes.
**************************************************************

As noted above, prioritization or de-selection of certain educational messages often depends on the prioritization or de-selection of other types of messages. Prioritization or de-selection of other message types (besides educational messages) is also often controlled by the placement of certain messages in one of the categories in Table I. These categorization of message type is set forth in Table II below.

TABLE II
Prioritizing and De-Selecting Messages in a Message Bank Where Messages Are
Differentiated by Message Type

Message types:

| | |
|---|---|
| (i) | Messages Recommending Patient Action |
| (ii) | Messages Recommending Data Input by Patient |
| (iii) | Messages Acknowledging Performance of Recommended Action or Input |
| (iv) | Educational Messages |

Messages of each type above are prioritized or de-selected based on placing a message in one or
more the categories set forth in Table I.
Placement of a particular message in one of the categories in Table I determines the placement of
certain other messages (of the same or of a different type) in one of the categories in Table I.
****************************************************************

As noted in Table II, placement of a particular message in one of the categories in Table I determines the placement of certain other messages (of the same or of a different type) in one of the categories in Table I. A number of exemplary messages of all four types in Table II are set forth in US Publ'n Nos. 20130035563 and 20120231431 (both incorporated by reference).

As a first example, certain educational messages will nearly always change their Table I category when another message type changes its Table I category. For example, when BG levels move far out of range (hyperglycemia or hypoglycemia), messages of type (iii) in Table II which praise the patient's actions will be absolutely de-selected (until the hyperglycemia or hypoglycemia is rectified). In such case, messages of type (i) specifying how to rectify the hyperglycemia or hypoglycemia will be prioritized, and educational messages (type (iv)) outlining the risks of hyperglycemia or hypoglycemia, as applicable, will also be prioritized. Other educational messages discussing the benefits of maintaining BG levels within the desired range may be concomitantly prioritized or de-selected.

Preferably, prioritizing and deselecting educational messages discussing the benefits of maintaining BG levels at desired the desired range is controlled by their effectiveness in accomplishing such objective. The effectiveness of educational messages can be determined using the weighting and re-weighting procedure set forth in FIGS. 1 to 5, or by other similar optimization procedures or other algorithms (readily apparent to those skilled in the art).

To determine long term effectiveness of educational messages on long term clinical outcomes or longer term control of indicators including BG level, one simply picks a greater value for “T” in FIGS. 1 to 5, and then re-weights. FIGS. 1 to 5 set forth an optimization process, where all messages are tested periodically. The last box in each of FIGS. 1 to 5 requires random selection of a message, though the messages in the message bank selected from have been weighted. This means that the less effective, lower weighted messages are still selected and sent, though at a lower frequency than messages with a higher weight.

The optimization of messages according to FIGS. 1 to 5 could be over the entire spectrum of users, or a subset thereof (based on criterion including education level, ethnicity, severity of disease, first language), or even for an individual—where the user is the only person the messages are optimized against, and the user's responses determine which messages are sent more frequently. Effectiveness of messages for an individual patient, or a sub-group of patients, can be determined by viewing only the messages sent to them and their response(s), under the process outlined in FIGS. 1 to 5. For individual optimization under the procedures in any of FIGS. 1 to 5, the number of users should be set at “1” for the user for whom the messages are being optimized.

The optimization process outlined in FIGS. 1 to 5 is a continuous prioritization and de-selection process, in which it is anticipated that effectiveness of messages can change over time; and therefore, their frequency changes to try to compensate for any decreasing or increasing effectiveness. Again, messages can then be optimized for a sub-group or an individual as noted above, if their effectiveness changes for such sub-group or individual.

The optimization process outlined in FIGS. 1 to 5 is a “pure optimization” embodiment, where iterative optimization (through weighting) controls the selection of all messages, based on message effectiveness. In a partial optimization embodiment, the optimization procedure would be used to determine message effectiveness, and then a message prioritization and de-selection procedure can be instituted to select and avoid certain messages, which are to be sent in connection with those messages found to be most effective. As an example of partial optimization, if a certain group of message are found best-suited for avoiding hypoglycemia through optimization, then other messages relating to avoiding hypoglycemia can be de-selected. In a pure optimization procedure, such other messages would receive lower weight and be sent less often than more effective messages, but would nevertheless be sent occasionally.

A partial optimization procedure can also be used where patient preferences are changed. As an example of such case, the messages which are most effective in prompting patient compliance with BG testing, diet or exercise regimens can be identified by an iterative optimization procedure. After the optimized messages are determined, they would be examined against the patient preferences, and those in conflict, would be de-selected. Similarly, certain messages which supported or were consistent with the user's preferences but which were not selected through optimization, could be prioritized.

In a partial optimization procedure, changing the frequency with which a message in categories (i), (ii) or (iii) of Table II is sent, generally brings about a change in sending frequency (through the optimization process or through prioritization or de-selection) of an educational message in category (iv) of Table II as well. In the case, for example, where a patient's preferences change, so that certain foods and exercise types are de-selected, certain educational messages (e.g., those touting the benefits of the de-selected foods or exercise types) can also be de-selected. Or, certain educational messages (e.g., those touting the benefits of doing more activity if the patient prefers to eat more carbohydrates) can be prioritized and sent at increased frequency.

A partial optimization procedure can include optimizing the frequency of sending of particular messages, where the optimal frequency is selected based on patient response. This means that sending certain messages at a specified frequency (not more or less than) leads to optimal patient responses. The responses can be measured over varying time periods, and optimization can be otherwise carried out as shown generally in FIGS. 1-5.

Other messages which appear to require “pure prioritization,” can in fact also account for patient preferences. Messages relating to administration of medication may be effectively fixed by prescription requirements. But in diabetes, many medications, including insulin, are administered in response to BG levels or patient feelings, meals and meal times and other indicia. Thus, messages relating to medication can, as a first step, be prioritized or de-selected in relation to such patient indicia and also, possibly, in relation to patient preferences. For example, patients may wish to administer insulin only at certain times of the day or only before or after meals.

Similarly, messages relating to patient-specific advice can be prioritized for that patient, and other messages can be conformed to that advice by prioritization or de-selection. The advice can be any action to reduce risk of morbidity (checking indicators or patient feelings) or control biometric indicators (including BG level) or increase patient well-being. The effectiveness of other messages can be optimized in view of the new message choices (after prioritization and de-selection), and in such case the optimization of such other messages is preferably individualized.

The specific methods, processes and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also noted that as used herein and in the appended claims, the singular forms “a,” “an,” and “the” include plural reference, and the plural include singular forms, unless the context clearly dictates otherwise. The term “messages” includes “message sets.” Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing, by Applicants. The invention has been described broadly and generically herein. Each of the narrower species and subgeneric groupings falling within the generic, disclosure also form part of the invention.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of increasing diabetic patient compliance with a recommended regime of diet, exercise, health-related checking or chemical indicator testing, wherein the patient is in an interactive system in which a glucometer carried by the patient communicates to a server which sends back messages from a message bank (containing at least 100 educational messages and at least 100 advisory messages) to the patient, and wherein advisory messages advise the patient to do one or more of: test for chemical or biochemical indicators, take action with respect to eating or exercising, and initiate a health-related check, and wherein educational messages advise the user of health risks and benefits based on test results or health-related check results or of the risks and benefits of action or inaction but do not recommend any action, the method comprising:
placing all messages in a messages bank;
associating certain advisory messages each with a different patient blood glucose level, so that when particular levels are received particular advisory messages are sent;
associating certain educational messages in the bank each with either a different patient blood glucose level or a different advisory message and wherein one or more of the following rules are applied:
(i) when a first particular patient blood glucose level is received a first particular educational message is also sent within a designated period and also sent a number of times thereafter at a specified frequency, or,
(ii) when a first particular advisory message is sent [a] the first particular educational message or another educational message is also sent within a designated period and also sent a number of times thereafter at a specified frequency, and
(iii) when the first particular patient blood glucose level is received a second particular advisory message is not sent for at least a designated period, or
(iv) when the first particular advisory message is sent a second particular educational message is not sent for at least a designated period;
providing the patient access to the system and receiving at the server blood glucose levels from the patient's glucometer and sending advisory and educational messages to the patient in accordance with rules (i) to (iv) above.

2. The method of claim 1 wherein advisory messages subject matter is either recommending patient action or recommending data input by patient of the results of a test for chemical or biochemical indicators.

3. The method of claim 1 further including sending messages acknowledging patient performance of recommended action or input.

4. The method of claim 1 wherein the association between particular blood glucose levels, and particular advisory messages and particular educational messages is based on factors including patient Hb1Ac level, ketone level, in vivo concentration of drugs or insulin, patient blood pressure, or patient gene expression, patient food and exercise preferences and exclusions; the patient's state of general health and co-morbidity risk; the patient's prescribed medications; the patient's previously reported blood glucose levels, diet, and exercise; the patient's previously reported health-related check results.

5. The method of claim 1 wherein the effectiveness of certain messages is determined based on how effective they are in causing patients to take actions which make their BG levels move into a desired range or in causing patients to take actions which maintain their BG levels in a desired range.

6. The method of claim 5 wherein the more effective messages are sent to patients more frequently than less effective messages.

7. The method of claim 5 wherein message frequency of sending to patient's device is governed by weighting the messages in the message bank in accordance with their effectiveness.

8. The method of claim 7 wherein the weighting and frequency of sending changes as the effectiveness of the message changes.

9. A method of increasing diabetic patient compliance with a recommended regime of diet, exercise, health-related checking or chemical indicator testing, wherein the patient is in an interactive system in which a glucometer carried by the patient communicates to a server which sends back messages from a message bank (containing (a) at least 100 educational messages, (b) at least 100 advisory messages, and (c) at least 10 messages acknowledging performance of an action recommended by a prior message to the patient) to the patient, and wherein advisory messages (type (b)) advise the patient to do one or more of: test for chemical or biochemical indicators, take action with respect to eating or exercising, and initiate a health-related check, and wherein educational messages (type (a)) advise the user of health risks and benefits based on test results or health-related check results or of the risks and benefits of action or inaction but do not recommend any action, the method comprising:
placing all messages in a message bank;
associating certain type (c) messages in the message bank each with a different patient blood glucose level or time-averaged level, so that after a particular blood glucose level or time-averaged level is received by the server, particular type (c) messages are sent;
associating certain advisory messages in the message bank each with a different patient blood glucose level, so that when particular levels are received particular advisory messages are sent;
associating certain educational messages in the message bank each with either a different patient blood glucose level or a different advisory message and wherein the following rules are applied:
(i) when a first particular patient blood glucose level is received a first particular educational message is also sent within a designated period and also sent a number of times thereafter at a specified frequency, or,
(ii) when a first particular advisory message is sent the first particular educational message or another educational message is also sent within a designated period and also sent a number of times thereafter at a specified frequency, and
(iii) when the first particular patient blood glucose level is received a second particular advisory message is not sent for at least a designated period, or
(iv) when the first particular advisory message is sent a second particular educational message is not sent for at least a designated period;
providing the patient access to the system and receiving at the server blood glucose levels from the patient's glucometer and sending advisory and educational messages to the patient in accordance with rules (i) to (iv) above; and
wherein the selection of the first particular advisory message, and the first or second particular educational message or another educational message, is based on the effectiveness of said messages in causing patients to take actions which make their BG levels move into a desired range or in causing patients to take actions which maintain their BG levels in a desired range.

10. The method of claim 9 wherein advisory messages subject matter is either recommending patient action or recommending data input by patient of the results of a test for chemical or biochemical indicators.

11. The method of claim 9 wherein the association between particular blood glucose levels, and particular advisory messages and particular educational messages is based on factors including patient Hb1Ac level, ketone level, in vivo concentration of drugs or insulin, patient blood pressure, or patient gene expression, patient food and exercise preferences and exclusions; the patient's state of general health and co-morbidity risk; the patient's prescribed medications;
the patient's previously reported blood glucose levels, diet, and exercise; the patient's previously reported health-related check results.

12. (canceled)

13. The method of claim 12 wherein the more effective messages are sent to patients more frequently than less effective messages.

14. The method of claim 12 wherein message frequency of sending to patient's device is governed by weighting the messages in the message bank in accordance with their effectiveness.

15. The method of claim 14 wherein the weighting and frequency of sending changes as the effectiveness of the message changes.

16. A method of increasing diabetic patient compliance with a recommended regime of diet, exercise, health-related checking or chemical indicator testing, wherein the patient is in an interactive system in which a glucometer carried by the patient communicates to a server which sends back messages from a message bank (containing (a) at least 100 educational messages, (b) at least 100 advisory messages, and (c) at least 10 messages acknowledging performance of an action recommended by a prior message to the patient) to the patient, and wherein advisory messages (type (b)) advise the patient to do one or more of: start or stop exertion, test for chemical or biochemical indicators, take action with respect to eating or exercising, and initiate a health-related check, and wherein educational messages (type (a)) advise the user of health risks and benefits based on test results or health-related check results or of the risks and benefits of action or inaction but do not recommend any action, the method comprising:
placing all messages in a message bank;
associating certain type (c) messages in the message bank each with a different patient blood glucose level or time-averaged level, so that after a particular blood glucose level or time-averaged level is received by the server, particular type (c) messages are sent;
associating certain advisory messages in the message bank each with a different patient blood glucose level, so that when particular levels are received particular advisory messages are sent;
associating certain educational messages in the message bank each with either a different patient blood glucose level or a different advisory message and wherein the following rules are applied:
(i) when a first particular patient blood glucose level is received a first particular educational message is also sent within a designated period and also sent a number of times thereafter at a specified frequency, or,
(ii) when a first particular advisory message is sent the first particular educational message or another educational message is also sent within a designated period and also sent a number of times thereafter at a specified frequency, and
(iii) when the first particular patient blood glucose level is received a second particular advisory message is not sent for at least a designated period, or
(iv) when the first particular advisory message is sent a second particular educational message is not sent for at least a designated period;
providing the patient access to the system and receiving at the server blood glucose levels from the patient's glucometer and sending advisory and educational messages to the patient in accordance with rules (i) to (iv) above; and
wherein the selection of the first particular advisory message, and the first or second particular educational message or another educational message, is based on the effectiveness of said messages in causing patients to take actions which make their BG levels move into a desired range or in causing patients to take actions which maintain their BG levels in a desired range; and wherein the more effective messages are sent to patients more frequently than less effective messages and wherein message frequency of sending to patients is governed by weighting the messages in the message bank in accordance with their effectiveness.

17. The method of claim 16 wherein the association between particular blood glucose levels, and particular advisory messages and particular educational messages is based on factors including patient Hb1Ac level, ketone level, in vivo concentration of drugs or insulin, patient blood pressure, or patient gene expression, patient food and exercise preferences and exclusions; the patient's state of general health and co-morbidity risk; the patient's prescribed medications; the patient's previously reported blood glucose levels, diet, and exercise; the patient's previously reported health-related check results.

18-20. (canceled)
